(19) United States
(12) Patent Application Publication
(10) Pub. No.: US 2003/0196921 A1
(43) Pub. Date: Oct. 23, 2003

(54) Simplified identification of sterilized equipment

(76) Inventor: Jane Giordano, Morristown, NJ (US)

Correspondence Address: Kenneth M. Goldman, 837 Ensenada Avenue, Berkeley, CA, 94707, US

(21) Appl. No.: 10/126,503

(22) Filed: Apr. 18, 2002

(51) Int. Cl. A61B 19/02

(52) U.S. Cl. 206/370; 206/363; 206/364; 206/438; 206/439; 206/459.1

(57) ABSTRACT

A set of sterilization bags is provided, each bag being capable of holding a multiplicity of medical instruments, wherein each bag comprises at least two sides affixed together to form a bag with an opening capable of being in an open position to receive medical instruments and a closed position for sterilization, and further wherein at least two bags in the set are differentiated by a feature in at least one side of each bag.

### TECHNICAL FIELD

The present invention relates to a method and system for simplified easy identification of sterilized instruments in a medical office or other setting.

### BACKGROUND OF THE INVENTION

Presently, medical offices employ sterilization bags to autoclave medical instruments. A single bag will normally contain more than one instrument, because all the instruments necessary for a single office procedure (an “instrument set-up”) are autoclaved together. For example, in a dental office, there are about five different procedures that are commonly performed. For a root canal procedure, there are five instruments in the instrument set-up. For a filling procedure, there is another, different instrument set-up that will go in another bag.

When all the instruments for a single procedure are bagged together, it is difficult to identify which procedure the instruments in a single bag relate to. Currently employed bags have a paper side and a clear tinted blue side. If the bags are all commingled in a drawer it is very difficult to quickly recognize the instrument set up you need. There is therefore a need in the art for an autoclave bagging system that will permit a medical practitioner to easily identify autoclaved instrument set-ups by set-up type.

### SUMMARY OF THE INVENTION

This invention solves the current problem with autoclave bagging systems by providing a system that will permit a medical practitioner to easily identify autoclaved instrument set-ups by set-up type.

In one aspect of this invention, a set of sterilization bags is provided, each bag being capable of holding a multiplicity of medical instruments, wherein each bag comprises at least two sides affixed together to form a bag with an opening capable of being in an open position to receive medical instruments and a closed position for sterilization, and further wherein at least two bags in the set are differentiated by a feature in at least one side of each bag.

### DETAILED DESCRIPTION OF THE INVENTION

Definitions

“Sterilization bag”, as used herein, means a flexible container which is capable of existing in open and closed states, and which can be placed in an autoclave. Accordingly, the bag is capable of remaining intact in a system that reaches high temperatures using steam to destroy all living microorganisms and their pathologic products.

“Feature”, as used herein, means a characteristic associated with a side of a sterilization bag that is capable of distinguishing one bag from another.

Sterilization Bag Set

The set of bags of the current invention are each capable of holding a multiplicity of medical instruments. The instruments may grouped together for any purpose. Preferably, the instruments are grouped together by medical procedure—that is, all of the instruments in a single bag may share the trait of being used for the same medical procedure. The medical procedure may be employed in any type of practice, for example orthopedic surgery, or dentistry. In a preferred embodiment of this invention, the instruments are used for dental procedures. The number of instruments in each bag may be any amount, although usually there will be from between two and 7 instruments in each bag.

Each bag is a flexible container which is capable of existing in an open and closed state. Preferably, the sterilization bag of the invention is comprised of two sheets of material attached and sealed at the edges, with one edge remaining open but capable of being sealed. The open edge is capable of receiving the instruments, capable of being sealed after receiving the instruments, and capable of being re-opened after autoclaving. In a preferred embodiment of the invention, the two sheets are rectangular or square, and attached on three edges. The material that comprises the bag sides must be capable of withstanding the high temperatures of the autoclave. Preferably, the sides are comprised of treated paper or plastic which are capable of withstanding high temperatures. The plastic may be transparent or opaque. In the most preferred embodiment of this invention, each bag is comprised of two rectangular sheets, one side made of treated paper, and the other of clear plastic.

The bags may have writing on one or more sides. In a preferred embodiment, the bags are comprised of a paper side and a plastic side and have a peel off strip on the open edge that folds on a perforated line. The paper side seals to the plastic side. Preferably, the writing will be in ink on the paper side. In another preferred embodiment, each bag has one or more arrows drawn on a side with a special ink which, when vapor or steam sterilized, turn dark brown. These arrows, when ethylene oxide processed (rather than vapor or steam processed), turn gold/brown.

An important characteristic of the sterilization bags that comprise the sterilization bag set is that the bags have a feature, and each bag in the bag set has a different example of that feature. For example, the color of at least one side of each bag is a preferred feature of this invention. In a preferred embodiment, at least one side of each bag in a bag set has a different color. Preferred colors are blue, pink, violet, green, and yellow. Shape, or writing are also potential features. Each bag in a bag set may have a different shape, or different writing.

What is claimed is:

1. A set of sterilization bags, each bag being capable of holding a multiplicity of medical instruments, wherein;
each bag comprises at least two sides affixed together to form a bag with an opening capable of being in an open position to receive medical instruments and a closed position for sterilization, and further wherein at least two bags in the set are differentiated by a feature in at least one side of each bag.

2. The set of sterilization bags of claim 1, where the differentiating feature is color.

3. The set of sterilization bags of claim 1 comprising at least five bags.

4. The set of sterilization bags of claim 3, wherein one side of each bag is made of a transparent material.

5. The set of sterilization bags of claim 4, where the transparent side of said at least five bags are colored green, pink, yellow, purple, and blue.
